(19) United States
(12) Patent Application Publication
(10) Pub. No.: US 2004/0186530 A1
(43) Pub. Date: Sep. 23, 2004

(54) Electrostimulating device

(76) Inventors: Fedorovich Sergey Gluschuk, Tomsk (RU); Semionovich Yacov Pekker, Tomsk (RU); Ivanovich Oleg Nalesnik, Tomsk (RU)

Correspondence Address: Angenehm Law Firm Ltd., P.O. Box 48755, Coon Rapids, MN, 55448-0755, US

(21) Appl. No.: 10/813,752

(22) Filed: Mar. 26, 2004

(30) Foreign Application Priority Data
Aug. 14, 2001 (RU) RU2001122940

(51) Int. Cl. A61N 1/18

(52) U.S. Cl. 607/40

(57) ABSTRACT

An electrostimulating device is provided, wherein one of the stimulating electrodes may be mobile and external to the casing. The mobile electrode is tethered to the device with an insulated conducting cable and is operative to increase the distance between the stimulating electrodes, so as to stimulate a greater volume of cells. Furthermore, an impedance modulator may be provided within the device, to sense the natural motor activity of the gastrointestinal tract by impedance variations and to modulate the electrostimulation, responsive to the impedance variations. Additionally, a galvanic cell may be provided within the device, using the natural gastrointestinal-tract fluid as a liquid medium, thus providing power for the device.

### FIELD OF THE INVENTION

The present invention relates generally to the field of electrostimulating medical devices, and particularly, to devices for electrostimulating internal tissue.

### BACKGROUND OF THE INVENTION

Medical devices for electrostimulating tissue of internal body cavities, such as the gastrointestinal tract, are known. Generally, an electrostimulating device includes a pulse generator and a power source, encapsulated within a biologically inert casing, and two stimulating electrodes, external to the casing. Electrostimulating devices may be used to promote motor activity, for example, of a weakly functioning gastrointestinal tract. They may also be used to as a post-surgical treatment. Additionally, they may be used as a prophylactic measure against ailments of the gastrointestinal tract, such as acute or chronic hypodynamia and adynamia.

Electrostimulating devices may be designed as ingestible capsules, or as implants. They may also be arranged on catheters.

Various forms of electrostimulating devices are described for example, in PCT Publications W09726042 and W097331679, Russian Inventor's Certificate 936931, and Russian Patents RU214O301, RU2103027, RU2128059, RU2097073, RU2104062, RU2134595, RU2121381, RU2135225, RU2107519, RU2129028, RU2153367, RU2153365, RU2071368, RU2066554, RU2071379, and RU1223922.

A drawback of known electrostimulating devices is the small distance between the electrodes, dictated by the design of the device, adapted for swallowing or for insertion to the body cavity. This reduces the volume of cells subject to the stimulation and increases spastic contractions of the walls of the stomach and the gastrointestinal tract. (Physiology of food digestion, L.: Nauka, 1974, 762 p.; Electronic equipment for organs and tissues stimulation, M.: Energoatomizdat, 1983, 384 p.; Berkingolit M. D., Glagoleva E. G. Electricity in living organisms, M.: Nauka, 1988; Muraviov L A. Technology of medicines, Moscow, Medicine, 1980, V. II, 583-592 p.)

Another drawback of known electrostimulating devices relates to the lack of synchronization with the rhythmic natural motor activity of the gastrointestinal tract, which is different among individuals, as well as in different sections of the gastrointestinal tract, and depends on the physical condition, the environment, and the phase of ingestion or starvation. Yet, known electrostimulating devices operate with stimulating pulses of a predetermined rate.

The asynchronous stimulation causes unpleasant and painful feelings and may depress the natural motor activity of the gastrointestinal tact and impair the evacuation function. (Gluchuk S. F. Autonomous electric simulators-probes of gastro-intestinal tract and system-complex approach of their design: Abstract of the thesis for a Ph.D. thesis in technical sciences: 05.11.17, M., 1989, 15 p; N. M. Lebedev. Biorhythms of digestive system, M.: Medicina, 1987/0 257 p; Popov O. S. Autonomous electric stimulation of gastro-intestinal tract in surgery: thesis for a Ph.D. thesis in technical sciences, Tomsk, 1988, 242 p; Hachatrian A.P. Clinical and pathophysiological aspects of electrical impedance measurement: thesis for a Ph.D. thesis in technical sciences: 14.00.16, 14.00.27, Tomsk, 1992, 52 p; TU KLGYa.431238.600 TU. Micro-scheme 1106A.—1991.-122p.

Additionally, the level of electrostimulation, like the rate, should be adapted to the individual and to his physical condition. But again, known electrostimulating devices operate with stimulating pulses of predetermined parameters, representing some average values for the population.

Another drawback of known electrostimulating devices relates to the power source, generally, a chemical battery, which becomes inoperative with time. When this happens, the device is discarded. (Autonomous implanted electric stimulator of connecting tissue, Pekarsky V. V., Gluchuk S. F., P. K. Shpilevsky, G. Z. Dambaiev, M. S. Derugina, Elektronnaya promishlennost, 1990, No. 12, 27 p.; Nalesnik 0.1., General theory of chemical origins of current, School-book, Tomst. P., house Kirov Politechnical Institute in Tomsk, 1985, 94 p.)

There is thus a widely recognized need for, and it would be highly advantageous to have an electrostimulating device devoid of the above limitations.

### SUMMARY OF THE INVENTION

In accordance with an aspect of the present invention, there is thus provided a device for electrostimulating tissue of internal body cavities, comprising:

a pulse generator, for generating electrostimulating pulses;

a power source, in electrical communication with the pulse generator;

a casing, which encapsulates and seals the pulse generator and the power source, within;

a first stimulating electrode, in electrical communication with the pulse generator, physically fixed to the casing and having a conducting surface, external to the casing, for making contact with the tissue; and

a second stimulating electrode, in electrical communication with the pulse generator, the second stimulating electrode being operative as a mobile electrode, wherein in a first conformation, adapted for insertion into the cavity, the second stimulating electrode is in close contact with the casing, and in a second conformation, adapted for electrostimulation. the second stimulating electrode is detached from the casing, tethered to the pulse generator, by an insulated conducting cable.

In accordance with an additional aspect of the present invention, the first conformation is maintained by an encapsulating material, which dissolves in the digestive system.

In accordance with another aspect of the present invention, the first conformation is maintained by glue, which is diluted in the digestive system.

In accordance with still another aspect of the present invention, the first conformation is maintained by a lubricant which is diluted in the digestive system.

In accordance with an additional aspect of the present invention, the device is adapted for ingestion.

In accordance with still another aspect of the present invention, the device is adapted for insertion via a catheter.

In accordance with an additional aspect of the present invention, the device includes a medication window, for applying medication to a specific location.

In accordance with yet an additional aspect of the present invention, the device includes an impedance modulator, for applying electrostimulation, which is proportional to measured tissue impedance, in synchrony with cycles of the tissue impedance.

In accordance with still an additional aspect of the present invention, the power source is a galvanic cell.

In accordance with yet an additional aspect of the present invention, the device includes a voltage converter, for converting voltage generated by the galvanic cell to an operating voltage for the pulse generator.

In accordance with still an additional aspect of the present invention, the insulated conducting cable by which the second stimulating electrode is tethered, is designed as a spring, which is in compression in the first conformation, and which maintains the first and second electrodes apart in the second conformation.

In accordance with another aspect of the present invention, there is thus provided a device for electrostimulating tissue of internal body cavities, comprising:

a pulse generator, for generating electrostimulating pulses;

a power source, in electrical communication with the pulse generator;

a casing, which encapsulates and seals the pulse generator and the power source, within;

first and second stimulating electrodes, in electrical communication with the pulse generator, and having conducting surfaces, external to the casing, for making contact with the tissue; and

an impedance modulator, comprising an impedance probe, for applying electrostimulation, which is proportional to measured tissue impedance, in synchrony with cycles of the tissue impedance.

In accordance with an additional aspect of the present invention, the device is adapted for ingestion.

In accordance with another aspect of the present invention, the device is adapted for implantation.

In accordance with still another aspect of the present invention, the device is adapted for insertion via a catheter.

In accordance with an additional aspect of the present invention, the device includes a medication window, for applying medication to a specific location.

In accordance with still an additional aspect of the present invention, the power source is a galvanic cell.

In accordance with yet an additional aspect of the present invention, the device includes a voltage converter, for converting voltage generated by the galvanic cell to an operating voltage for the pulse generator.

In accordance with still an additional aspect of the present invention, the first stimulating electrode, is physically fixed to the casing, and the second stimulating electrode, is operative as a mobile electrode, wherein in a first conformation, adapted for insertion into the cavity, the second stimulating electrode is in close contact with the casing, and in a second conformation, adapted for electrostimulation, the second stimulating electrode is detached from the casing, tethered to the pulse generator, by an insulated conducting cable.

In accordance with yet an additi6nal aspect ofthe present invention, the insulated conducting cable by which the second stimulating electrode is tethered, is designed as a spring, which is in compression in the first confirmation, and which maintains the first and second electrodes apart in the second conformation.

In accordance with still another aspect of the present invention, there is thus provided a device for electrostimulating tissue of internal body cavities, comprising:

a pulse generator, for generating electrostimulating pulses;

first and second stimulating electrodes, in electrical communication with the pulse generator, and having conducting surfaces, adapted for making contact with the tissue;

a casing, which forms a first chamber, which encapsulates and seals the pulse generator from body fluids; and

a power source, formed as a galvanic cell, for powering the pulse generator.

In accordance with an additional aspect ofthe present invention, the galvanic cell comprises:

an inner space, formed as a second chamber of the casing, the second chamber having portholes for admitting body fluids to the inner space; and

a third electrode, located within the inner space, having a different electrochemical potential from the first electrode, and forming an electrical circuit with the first electrode, the circuit being closed by body fluids within the inner space.

In accordance with another aspect of the present invention, the galvanic cell comprises:

an inner space, formed as a second chamber of the casing, the second chamber having portholes for admitting body fluids to the inner space; and

third and fourth electrodes, located within the inner space, having a different electrochemical potential between them, and timing an electrical circuit, the circuit being closed by body fluids within the inner space.

In accordance with an additional aspect of the present invention, the device includes a voltage converter, for converting voltage generated by the galvanic cell to an operating voltage for the pulse generator.

In accordance with still an additional aspect of the present invention, the device is adapted for ingestion.

In accordance with another aspect of the present invention, the device is adapted for implantation.

In accordance with still another aspect of the present invention, the device is adapted for insertion via a catheter.

In accordance with an additional aspect of the present invention, the device includes a medication window for applying medication to a specific location.

In accordance with still an additional aspect of the present invention, the first stimulating electrode, with which the galvanic cell is formed, is physically fixed to the casing, and the second stimulating electrode, is operative as a mobile electrode, wherein in a first conformation, adapted for insertion into the cavity, the second stimulating electrode is in close contact with the casing, and in a second conformation, adapted for electrostirnulation, the second stimulating electrode is detached from the casing, tethered to the pulse generator, by an insulated conducting cable.

In accordance with yet an additional aspect ofthe present invention, the insulated conducting cable by which the second stimulating electrode is tethered, is designed as a spring, which is in compression in the first conformation, and which maintains the first and second electrodes apart in the second conformation.

In accordance with still an additional aspect of the present invention, the device includes an impedance modulator, for applying electrostimulation, which is proportional to measured tissue impedance, in synchrony with cycles of the tissue impedance.

In accordance with an aspect of the present invention, there is thus provided a method for electrostimulating tissue of internal body cavities, comprising:

employing a device for electrostimulating tissue of internal body cavities, which includes;

a pulse generator, for generating electrostimulating pulses;

a power source, in electrical communication with the pulse generator;

a casing, which encapsulates and seals the pulse generator and the power source, within;

a first stimulating electrode, in electrical communication with the pulse generator, physically fixed to the casing and having a conducting surface, external to the casing, for making contact with the tissue; and

a second stimulating electrode, in electrical communication with the pulse generator, the 20 second stimulating electrode being operative as a mobile electrode, wherein in a first conformation, adapted for insertion into the cavity, the second stimulating electrode is in close contact with the casing, and in a second conformation, adapted for electrostimulation, the second stimulating electrode is detached from the casing, tethered to the pulse generator, by an insulated conducting cable;

inserting the device into the cavity in the first conformation, wherein the second stimulating electrode is in close contact with the casing; and

electrostimulating the tissue in the second conformation, wherein the second stimulating electrode is detached from the casing, tethered to the pulse generator, by the insulated conducting cable.

In accordance with another aspect of the present invention, there is thus provided a method for electrostimulating tissue of internal body cavities comprising:

employing a device for electrostimulating tissue of internal body cavities, which includes:

a pulse generator, for generating electrostimulating pulses;

a power source, in electrical communication with the pulse generator, a casing, which encapsulates and seals the pulse generator and the power source, within;

first and second stimulating electrodes, in electrical communication with the pulse generator, and having conducting surfaces, external to the casing, for making contact with the tissue; and

an impedance modulator, comprising an impedance probe, for applying electrostimulation, which is proportional to measured tissue impedance, in synchrony with cycles of the tissue impedance; measuring tissue impedance; and

Electrostimulating the tissue, wherein the electrostimulation is proportional to measured tissue impedance, in synchrony with cycles of the tissue impedance.

In accordance with still another aspect of the present invention, there is thus provided a method for electrostimulating tissue of internal body cavities, comprising:

employing a device for electrostimulating tissue of internal body cavities, which includes:

a pulse generator, for generating electrostimulating pulses;

first and second stimulating electrodes, in electrical communication with the pulse generator, and having conducting surfaces, adapted for making contact with the tissue;

a casing, which forms a first chamber, which encapsulates and seals the pulse generator from body fluids; and

a power source, formed as a galvanic cell, for powering the pulse generator;

powering the pulse generator by the galvanic cell; and

electrostimulating the tissue.

The present invention successfully addresses the shortcomings of the presently known configurations by providing an electrostimulating device, wherein one of the stimulating electrodes may be mobile and external to the casing. The mobile electrode is tethered to the device with an insulated conducting cable and is operative to increase the distance between the stimulating electrodes, so as to stimulate a greater volume of cells. Furthermore, an impedance modulator may be provided within the device, to sense the natural motor activity of the gastrointestinal tract by impedance variations and to modulate the electrostimulation, responsive to the impedance variations. Additionally, a galvanic cell may be provided within the device, using the natural gastrointestinal-tract fluid as a liquid medium, thus providing power for the device.

### BRIEF DESCRIPTION OF THE DRAWINGS

The invention is herein described, by way of example only, with reference to the accompanying drawings. With specific reference to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In, the drawings:

FIGs. 1A-1B are schematic illustrations of an electrostimulating device, comprising a mobile electrode, in accordance with a preferred embodiment of the present invention;

FIG. 2 is a schematic illustration of an electrostimulating device, comprising an impedance modulator, in accordance with a preferred embodiment of the present invention;

FIGS. 3A-3C are schematic illustrations of the operation of the impedance modulator of FIG. 2, in accordance with a preferred embodiment of the present invention;

FIG. 4 is a block diagram the impedance modulator of FIG. 2, in accordance with a preferred embodiment of the present invention;

FIG. 5 is a schematic illustration of an electrostimulating device, comprising a galvanic cell, in accordance with a preferred embodiment of the present invention;

FIGS. 6A and 6B are block diagrams of galvanic cells, in accordance with preferred embodiments of the present invention;

FIGS. 7A and 7B are schematic illustrations of electrostimulating implant devices, in accordance with preferred embodiments of the present invention; and

FIG. 8 is a schematic illustration of an electrostimulating device, arranged on a catheter, in accordance with a preferred embodiment of the present invention

### DESCRIPTION OF THE PREFERRED EMBODIMENTS

The present invention is of an electrostimulating device, wherein one of the stimulating electrodes may be mobile and external to the casing. The mobile electrode is tethered to the device with an insulated conducting cable and is operative to increase the distance between the stimulating electrodes, so as to stimulate a greater volume of cells. Furthermore, an impedance modulator may be provided within the device, to sense the natural motor activity of the gastrointestinal tract by impedance variations and to modulate the electrostimulation, responsive to the impedance variations. Additionally, a galvanic cell may be provided within the device, using the natural gastrointestinal-tract fluid as a liquid medium, thus providing power for the device.

The principles and operation of the systems and methods according to the present invention may be better understood with reference to the drawings and accompanying descriptions.

Before explaining at least one embodiment of the invention in detail, it is to be understood that the Invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

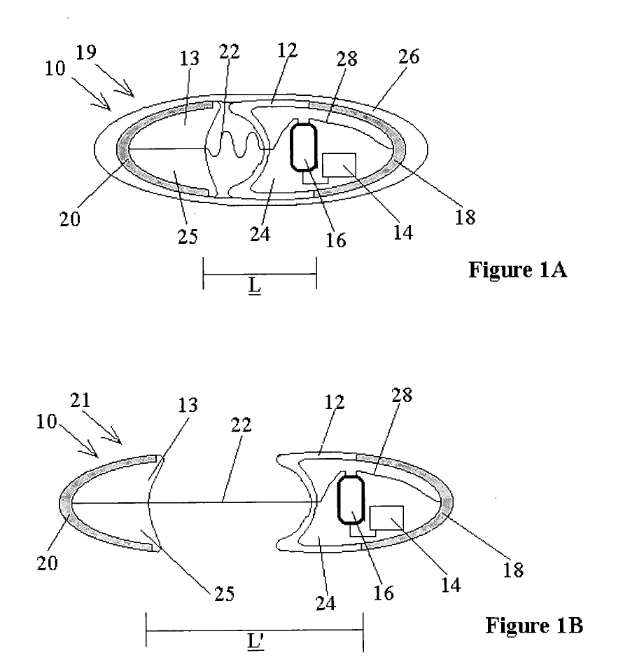

Referring now to the drawings, FIGS. 1A-1B are schematic illustrations of an electrostimulating device 10 comprising a mobile electrode 20, in accordance with a referred embodiment of the present invention, Preferably, device 10 is an electrostimulating ingestible capsule, hiving a first conformation 19, adapted for ingestion (FIG. 1A), and a second conformation 21, wherein mobile electrode 20 is deployed (FIG. 1B).

As seen in FIG. 1A, electrostimulating device 10 includes a power source 14, a pulse generator 16, connecting wires 28, and first and second stimulating electrodes 18 and 20, which are preferably semi-spherical in shape. Additionally, electrostimulating device 10 includes a casing 12, preferably biologically inert and dielectric. Casing 12 and first electrode 18 enclose a sealed first assembly 24 of electrostimulating device 10.

In accordance with the present embodiment, second stimulating electrode 20 is designed as a mobile electrode, being external to first assembly 24, tethered by an insulated conducting cable 22. Insulated conducting cable 22 may be formed as a spring 22 in compression, acting as an Insulated conducting element, between mobile electrode 20 and pulse generator 16.

Mobile electrode 20 may he mounted on a dielectric material 13. Together, mobile electrode 20 and dielectric material 13 form a sealed second assembly 25.

In first conformation 19, adapted for ingestion, first and second assemblies 24 and 25 may be enclosed within a soluble capsule 26, formed, for example, of a pharmaceutical gelatin or another easily soluble material. In addition to retaining first and second assemblies 24 and 25 together, capsule 26 acts as an insulator between electrodes 18 and 20 and the tissue, until dissolving in the digestive system. Additionally, or alternatively, first and second assemblies 24 and 25 may be glued together, for example, by KGS-IOMO.250.010.001 TI lubricant glue, which is diluted in the digestive system.

Alternatively, a biologically neutral lubricant such as Vaseline may be used as glue. In the digestive system, the Vaseline is diluted.

Additionally, in first conformation 19, adapted fir ingestion, electrostimulating device 10 may include a candy-like coating, formed, for example, of crusted sugar, sugared gelatin, chocolate, or the like, acting as a lubricant and (or) as an insulator, for making electrostimulating device 10 somewhat palatable.

The overall size of electrostimulating device 10, in first conformation 19, adapted for ingestion, may be about 2 cm in length, and about 1 cm in width. It will be appreciated that smaller dimensions are possible. Additionally, somewhat larger dimensions may be possible. Preferably, a distance L between electrodes 18 and 20 is between 0.5 and 1.0 cm. It will be appreciated that other distances, which may be smaller or larger, are also possible.

As seen in FIG. 1B. in the gastrointestinal tract, electrostimulating device 10 assumes second conformation 21, as encapsulating material 26 is dissolved, or the glue or lubricant is diluted, and mobile electrode 20 is deployed. Preferably, a distance L′ between first and second stimulating electrodes 18 and 20. in second conformation 21, is between 1.5 and 2.5 cm. It will be appreciated that other distances, which may be smaller or larger, are also possible.

Biologically inert, dielectric casing 12 may be formed, for example, of polycarbonate, polyethylene, natural rubber, silicon, or a composite formed for example, as an epoxy resin impregnated with glass fibers.

Stimulating electrodes 18 and 20 may be formed of an alloy, such as stainless steel, for example, 12X18H9, or of a cobalt-nickel-chrome alloy 4OK27HXMP, or 4OK27HXMPa, or of another biologically compatible conducting metal or alloy.

In accordance with alternate embodiments of die present invention, electrodes 25 18 and 20 may be spherical, elliptical, or of other shapes. Additionally, electrodes 18 and 20 need not be of the same shape.

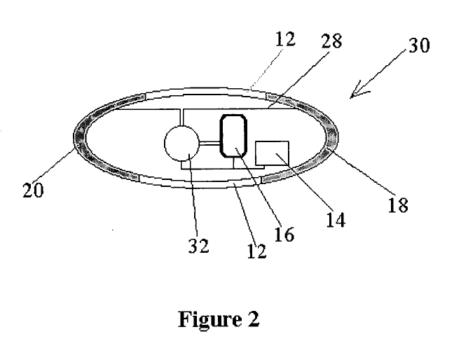

Referring further to the drawings, FIG. 2 is a schematic illustration of an electrostimulating device 30, comprising an impedance modulator 32, in accordance with a preferred embodiment of the present invention.

According to Hachatrian (Hachatrian A. P., Clinical and pathophysiological aspects of electrical impedance measurement: thesis for a Ph.D., thesis in technical sciences: 14.00.16, 14.00.27, Tomsk, 1992, 52 p.) electric impedance of tissue of the gastrointestinal tract depends on the extent of muscles contraction. Low frequency current (between several hundred Hz to several thousand Hz) flows in the tissue mainly through intercellular gaps. Resistance to this current is determined by the intercellular dimensions. When muscles contract, the intercellular size is reduced and the resistance is increased. Therefore, measured impedance values are indicative of the extent of muscle contraction, and stimulated pulses that are proportional to the measured impedance values will be synchronized with the muscle action in different gastrointestinal-tract sections.

Preferably, device 30 is en electrostimulating ingestible capsule, which includes casing 12, power source 14, pulse generator 16, first and second stimulating electrodes 18 and 20, and wiring 28. Casing 12 is biologically inert and dielectric. First and second stimulating electrodes 18 and 20 may be semi-spherical ira shape. imbedded in casing 12.

Additionally, electrostimulating device 30 includes impedance modulator 32, which interfaces between pulse-generator 16 one the one hand, and electrodes 18 and 20, on the other. impedance modulator 32 is operative to measure tissue impedance and to modulate the electrostimulation so that it is proportional to tissue impedance at any time, thus synchronized with the cycles of tissue impedance. Preferably, pulse generator 16 and electrodes 18 and 20 are used both for measuring tissue impedance, and for electrostirnulation, responsive to the impedance measurements.

In accordance with the present invention, electrostimulating device 30 may also be hand-held, for example during surgery.

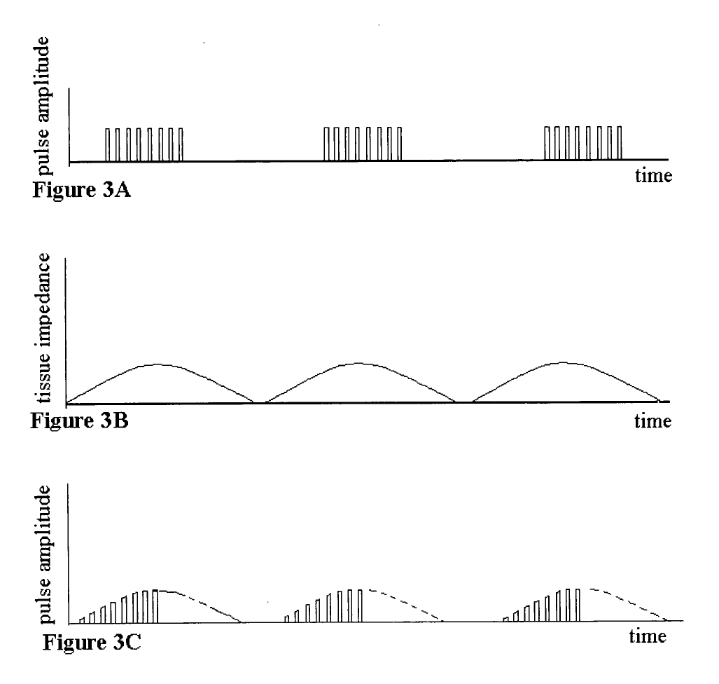

Referring further to the drawings, FIGS. 3A-3C illustrate the operation of impedance modulator 32, in accordance with a preferred embodiment of the present invention.

FIG. 3A illustrates a mode of electrostimulation, based on average values of electrostimulating pulses, used in the absence of impedance modulator 32.

FIG. 3B illustrates measured impedance signals, as a function of time, indicative of the rhythmic natural motor activity of the tissue, such as the gastrointestinal tract.

FIG. 3C illustrates a mode of electrostimulation, based on electrostimulating pulses, which are proportional to the measured tissue impedance, thus synchronized with the tissue impedance cycle, in accordance with a preferred embodiment of the present invention.

Preferably, as seen in FIG. 3C, the electrostirnulating pulses arc produced in series. Preferably, each series begins substantially with tissue contraction, and increases in amplitude, to a maximum that occurs at about maximum tissue contraction.

Additionally, when no tissue contraction is observed, the mode of electrostimulation of FIG. 3A, based on average values of electrostimulating pulses, is used. Furthermore, when the tissue undergoes periods of contractions and periods of no contractions, the mode of electrostimulation is switched between that seen in FIG. 3A and that seen in FIG. 3C.

It should be pointed out that when moving along the gastrointestinal tract, electrostimulating device 30 may not always accurately reflect impedance variations of the gastrointestinal tract walls. However, in a case of pronounced disorder of the intestinal tract motility, electrostimulating device 30 will remain motionless for a time, and synchronization of the electrostimulating pulses with the natural motor activity of the gastrointestinal tract walls is likely to take place.

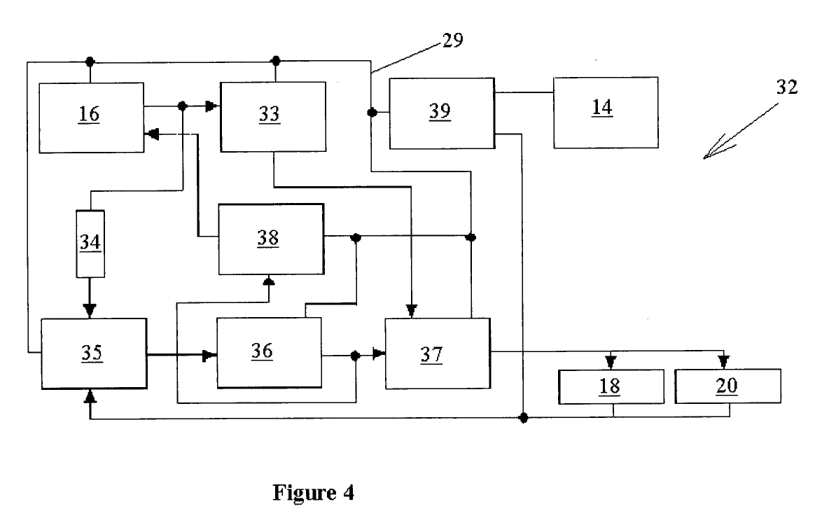

Referring further to the drawings, FIG. 4 is a diagram of impedance modulator 32, in accordance with a preferred embodiment of the present invention.

In the absence of impedance modulator 32, pulse generator 16 generates pulses of predetermined amplitude and duration, at a predetermined rate, as seen in FIG. 3A. However, impedance modulator 32 is operative to convert these into pulse pairs. The first pulse of the pulse pair is used for measuring tissue impedance and the second pulse of the pulse pair is used for electrostimulation

Preferably, impedance modulator 32 includes the following components:

1. a restricting resistor 34, for decreasing the amplitude ofthe first 30 pulses ofthe pulse pairs, by at least two orders of magnitude, so as not to cause electrostimulation during impedance measurement.

2. an impedance measuring unit 35, for evaluating tissue impedance, based on excitation of electrodes 18 and 20, by the low-amplitude first pulses, preferably at the frequency of pulse generator 16, preferably, at 200 Hz;

3. an amplifier 36, which amplifies the measured impedance signals;

4. an output device 37, for shaping the second pulses of the pulse pairs, arriving from pulse generator 16, via a frequency divider 33, so that the second pulses are proportional to the amplified impedance signals arriving from amplifier 36;

5. a synchronization unit 38, for synchronizing the shaped second pulses arriving from output device 37, with the cycles of measured tissue impedance; and

6. a switch 39, connecting power source 14 and a common bus 29.

It will be appreciated that impedance modulator 32 may be arranged somewhat differently. For example, impedance modulator 32 may include a microprocessor.

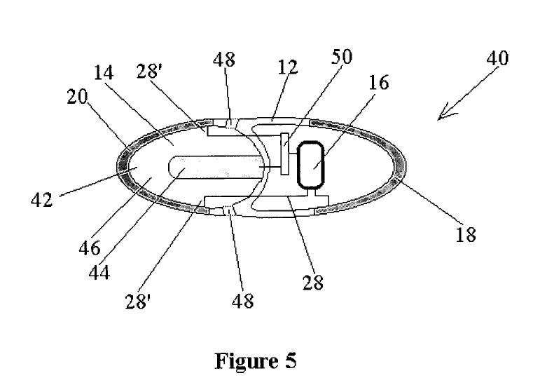

Referring further to the drawings, FIG. 5 is a schematic illustration of an electrostimulating device 40, comprising a galvanic cell 42, in accordance with a preferred embodiment of the present invention.

Preferably, device 40 is an electrostimulating ingestible capsule, which includes casing 12, pulse generator 16, first and second stimulating electrodes 18 and 20, and wiring 28. Casing 12 is biologically inert and dielectric. First and second stimulating electrodes 18 and 20 may be semi-spherical in shape, imbedded in casing 12.

In accordance with a preferred embodiment of the present invention, power source 14 comprises galvanic cell 42. Thus, electrostimulating device 40 includes a third electrode 44, formed of a metal or alloy which has a different electro-chemical potential than stimulating electrode 20, so as to form a galvanic cell with electrode 20. Preferably electrode 44 serves as the anode. For example, electrode 44 may be formed of aluminum, zinc, or magnesium, or alloys thereof, while electrode 20 (like electrode 18 ) may be formed of stainless steel, such as 12X18H9, or of cobalt-nickel-chrome alloy, such as 4OK27HXMP, or 4OK27HXMPa.

Galvanic cell 42 includes portholes 48, for admitting body fluids, such as gastrointestinal fluids, into an inner space 46, for acting as the galvanic cell liquid medium. Inner space 46 may further include a porous substance (not shown), such as a non-woven material. Preferably, a voltage converter 50 converts the voltage generated by galvanic cell 42 to operating voltage for electrostimulation, which is fed to pulse generator 16. Voltage converter 50 may be, for example, a multiplier connected in series with a voltage stabilizer.

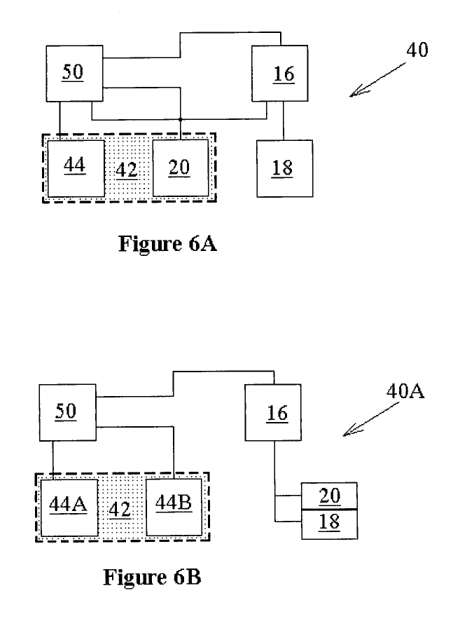

Referring further to the drawings, FIG. 6A is a block diagram of the galvanic cell of electrostimulating device 40 (FIG. 5), in accordance with a preferred embodiment of the present invention. Voltage generated between electrodes 44 and 20 is converted to operating voltage by voltage converter 50 and fed to pulse generator 16, which generates pulses that are fed to electrodes 18 and 20, for electrostimulation of the tissue.

Referring further to the drawings, FIG. 6B is a block diagram of another galvanic cell of an electrostimulating device 40A, wherein a fourth electrode is used as a cathode, in place of stimulating electrode 20, in accordance with another preferred embodiment of the present invention. Voltage generated between two electrodes 44A and 44B is converted to operating voltage by voltage converter 50 and fed to pulse generator 16, which generates pulses that are fed to electrodes 18 and 20, for electrostimulation of the tissue.

Experimentally measured voltages for different pair of electrode materials in 1%-hydrochloric acid, which is similar to the digestive juices, at a discharge current of 1 mA, are given in Table 1. A current of 1 mA is between one and two orders of magnitude greater than the average working current of device 40, for electrostimulation.

TABLE 1

| ELECTRODE PAIR | OPERATING VOLTAGE, V |
|---|---|
| Al and SS-12X18H9 | 0.82-0.98 |
| Zn and SS-12X18H9 | 0.36-0.4 |
| Mg and SS-12X18H9 | 1.24-1.3 |

It will be appreciated that the voltage produced by galvanic cell 42, as shown in the examples given in Table 1, is sufficient for operating device 40.

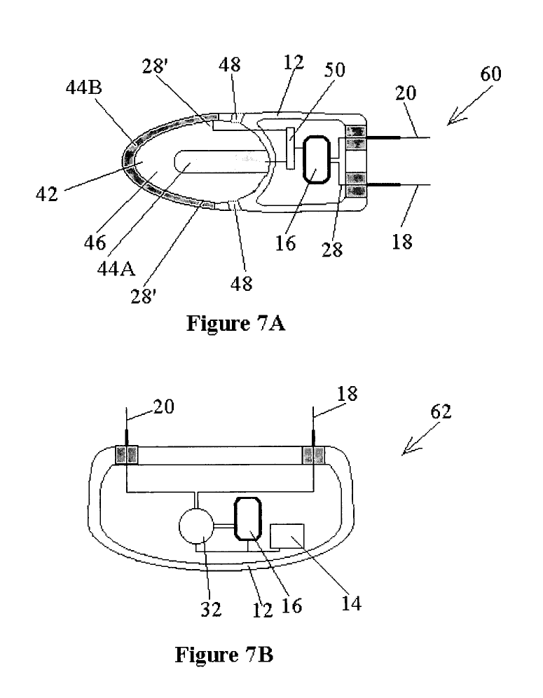

Referring further to the drawings, FIGS. 7A and 7B are schematic illustrations of an electrostimulating implant devices 60 and 62, in accordance with preferred embodiments of the present invention.

As seen in FIG. 7A, implant device 60 includes wire electrostimulating electrodes 18 and 20, pulse generator 16, voltage converter 50 and galvanic cell 42, comprising anode 44A, cathode 44B, and inner space 46 between them.

As seen in FIG. 7B, implant device 62 includes wire electrostimulating electrodes 18 and 20, pulse generator 16, power source 14, and impedance modulator 32, for applying electrostimulation, which is proportional to measured tissue impedance, in synchrony with cycles of the tissue impedance, in a manner analogous to that of electrostimulating device 30 (FIG. 2).

In accordance with the present invention, wire electrostimulating electrodes 18 and 20 may be used as studs or screws for anchoring implant device 60 or 62 in the tissue. Wire electrostimulating electrodes 18 and 20 may be formed of an alloy such as 4OK27HXMPa, which is easily reshaped in accordance with the constrains of the stimulation area. Alternatively, any other known implanting means may be used. Implant devices 60 or 62 may be implanted by surgery, or with a catheter.

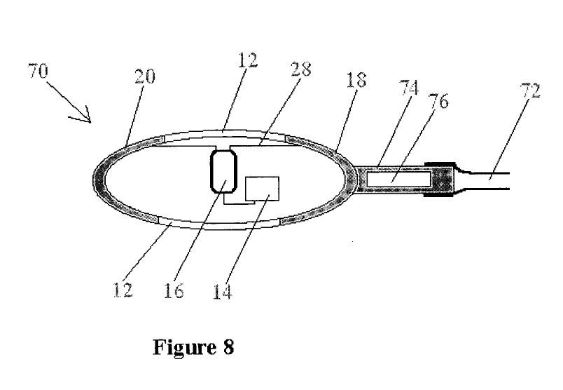

Referring further to the drawings. FIG. 8 is a schematic illustration of an electrostimulating device 70, mounted on a catheter 72, in accordance with another preferred embodiment of the present invention. Catheter 72 may be used for inserting electrostimulation device 70, to the gastrointestinal tract, for example, when there may be a blockage in the gastrointestinal tract, so that an ingestible capsule may not be used, or when treatment of only a specific portion of the gastrointestinal tract is desired. Additionally, electrostimulation device 70 may be used to treat body cavities other than the gastrointestinal tract.

Electrostimulating device 70 preferably includes an adapter 74, for mounting on catheter 72. Additionally, adapter 74, or catheter 72 may further include a medication window 76 for applying medication at a specific location. Preferably, window 76 may be opened extra-corporeally, thus applying the medication at a target location.

Electrostirnulating device 70 may further include mobile electrode 20 (FIGS. 1A-1B), impedance modulator 32 (FIG. 2) and (or) galvanic cell 42 (FIG. 5).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for electrostimulating tissue of internal body cavities, comprising;
a pulse generator, for generating electrostimulating pulses;
a power source, in electrical communication with said pulse generator;
a casing, which encapsulates and seals said pulse generator and said power source, within;
a first stimulating electrode, in electrical communication with said pulse generator, physically fixed to said casing and having a conducting surface, external to said casing, for making contact with said tissue; and
a second stimulating electrode, in electrical communication with said pulse generator, said second stimulating electrode being operative as a mobile electrode, wherein in a first conformation, adapted for insertion into said cavity, said second stimulating electrode is in close contact with said casing, and in a second conformation, adapted for electrostimulation, said second stimulating electrode is detached from said casing, tethered to said pulse generator, by an insulated conducting cable.

2. The device of claim 1, wherein said first conformation is maintained by an encapsulating material, which dissolves in the digestive system.

3. The device of claim 1, wherein said firs: conformation is maintained by glue, which is diluted in the digestive system.

4. The device of claim 1, wherein said first conformation is maintained by a lubricant which is diluted in the digestive system.

5. The device of claim 1, wherein said device is adapted for ingestion.

6. The device of claim 1, wherein said device is adapted for insertion via a catheter.

7. The device of claim 6, wherein said device further includes a medication window, for applying medication to a specific location.

8. The device of claim 1, and Rather including an impedance modulator, for applying electrostimulation, which is proportional to measured tissue impedance, in synchrony with cycles of said tissue impedance.

9. The device of claim 1, wherein said power source is a galvanic cell.

10. The device of claim 9, and further including a voltage converter, for converting voltage generated by said galvanic cell to an operating voltage fir said pulse generator.

11. The device of claim 1, wherein said insulated conducting cable by which said second stimulating electrode is tethered, is designed as a spring, which is in compression in said first conformation, and which maintains said first and second electrodes apart in said second conformation.

12. A device for electrostimulating tissue of internal body cavities, comprising:
a pulse generator, for generating electrostimulating pulses;
a power source, in electrical communication with said pulse generator;
a casing, which encapsulates and seals said pulse generator and said power source, within;
first and second stimulating electrodes, in electrical communication with said pulse generator, and having conducting surfaces, external to said casing, for making contact with said tissue; and
an impedance modulator, comprising an impedance probe, for applying electrostimulation, which is proportional to measured tissue impedance, in synchrony with cycles of said tissue impedance.

13. The device of claim 12, wherein said device is adapted for ingestion.

14. The device of claim 12, wherein said device is adapted for implantation.

15. The device of claim 12, wherein said device is adapted for insertion via a catheter.

16. The device of claim 15, wherein said device further includes a medication window, for applying medication to a specific location.

17. The device of claim 12, wherein said power source is a galvanic cell.

18. The device of claim 17, and further including a voltage converter, for converting voltage generated by said galvanic cell to an operating voltage for said pulse generator.

19. The device of claim 12, wherein said first stimulating electrode, is physically fixed to said casing, and wherein said second stimulating electrode, is operative as a mobile electrode, wherein in a first conformation, adapted for insertion into said cavity, said second stimulating electrode is in close contact with said casing, and in a second conformation, adapted for electrostimulation, said second stimulating electrode is detached from said casing, tethered to said pulse generator, by an insulated conducting cable.

20. The device of claim 19, wherein said insulated conducting cable by which said second stimulating electrode is tethered, is designed as a spring, which is in compression in said first conformation, and which maintains said first and second electrodes apart in said second conformation.

21. A device for electrostimulating tissue of internal body cavities, comprising:
a pulse generator, for generating electrostimulating pulses;
first and second stimulating electrodes, in electrical communication with said pulse generator, and having conducting surfaces, adapted for making contact with said tissue;
a casing, which forms a first chamber, which encapsulates and seals said pulse generator from body fluids; and
a power source, fanned as a galvanic cell, for powering said pulse generator.

22. The device of claim 21, wherein said galvanic cell comprises:
an inner space, formed as a second chamber of said caning, said second chamber having portholes far admitting body fluids to said inner space; and
a third electrode, located within said inner space, having a different electrochemical potential from said first electrode, and forming an electrical circuit with said first electrode, said circuit being closed by body fluids within said inner space.

23. The device of claim 21, wherein said galvanic cell comprises:
an inner space, formed as a second chamber of said casing, said second chamber having portholes for admitting body fluids to said inner space; and
third and fourth electrodes, located within said inner space, having a different electrochemical potential between tern, and Conning an electrical circuit, said circuit being closed by body fluids within said inner space.

24. The device of claim 21, and further including a voltage converter, for converting voltage generated by said galvanic cell to an operating voltage for said pulse generator.

25. The device of claim 21, wherein said device is adapted for ingestion.

26. The device of claim 21, wherein said device is adapted for implantation.

27. The device of claim 21, wherein said device is adapted for insertion via a catheter.

28. The device of claim 27, wherein said device farther includes a medication window, for applying medication to a specific location.

29. The device of claim 21, wherein said first stimulating electrode, with which said galvanic cell is formed, is physically fixed to said casing, and wherein said second stimulating electrode, is operative as a mobile electrode, wherein in a first conformation, adapted for insertion into said cavity, said second stimulating electrode is in close contact with said casing, and in a second conformation, adapted for electrostimulation, said second stimulating electrode is detached front said casing, tethered to said pulse generator, by an insulated conducting cable.

30. The device of claim 29, wherein said insulated conducting cable by which said second stimulating electrode is tethered, is designed as a spring, which is in compression in said first conformation, and which maintains said first and second electrodes apart ira said second conformation.

31. The device of claim 21, and further including an impedance modulator, for applying electrostimulation, which is proportional to measured tissue impedance, in synchrony with cycles of said tissue impedance.

32. A method for electrostirnulating tissue of internal body cavities, comprising:
employing a device for electrostimulating tissue of internal body cavities, which includes:
a pulse generator, for generating electrostimulating pulses;
a power source, in electrical communication with said pulse generator;
a casing, which encapsulates and seals said pulse generator and said power source, within;
a first stimulating electrode, in electrical communication with said pulse generator, physically fixed to said casing and having a conducting surface, external to said casing, for making contact with said tissue; and
a second stimulating electrode, in electrical communication with said pulse generator, said second stimulating electrode being operative as a mobile electrode, wherein in a first conformation, adapted for insertion into said cavity, said second stimulating electrode is in close contact with said casing, and in a second conformation, adapted for electrostimulation, said second stimulating electrode is detached from said casing, tethered to said pulse generator, by an insulated conducting cable;
inserting said device into said cavity in said first conformation, wherein said second stimulating electrode Is in close contact with said casing; and
electrostirnulating said tissue in said second conformation, wherein said second stimulating electrode is detached from said casing, tethered to said pulse generator, by said insulated conducting cable.

33. The method of claim 32, wherein said first conformation is maintained by an encapsulating material, which dissolves in the digestive system.

34. The method of claim 32, wherein said first conformation is maintained by glue, which is diluted in the digestive system.

35. The method of claim 32, wherein said first conformation is maintained by a lubricant which is diluted in the digestive system.

36. The method of claim 32, wherein said device is adapted for ingestion.

37. The method of claim 32, wherein said device is adapted for insertion via a catheter.

38. The method of claim 37, and further including applying medication via said catheter.

39. The method of claim 32, wherein said device further includes an impedance modulator, for applying electrostimulation, which is proportional to measured tissue impedance, in synchrony with cycles of said tissue impedance.

40. The method of claim 32, wherein said power source is a galvanic cell.

41. The method of claim 40, and further including converting voltage generated by said galvanic cell to an operating voltage fir said pulse generator.

42. The method of claim 32, wherein said Insulated conducting cable by which said second stimulating electrode is tethered, is designed as a spring, which is in compression in said first conformation, and which maintains said first and second electrodes apart in said second conformation.

43. A method for electrostimulating tissue of internal body cavities, comprising:
employing a device for electrostirnulating tissue of internal body cavities, which includes;
a pulse generator, for generating electrostimulating pulses;
a power source, in electrical communication with said pulse generator;
a casing, which encapsulates and seals said pulse generator and said power source, within;
first and second stimulating electrodes, in electrical communication with said pulse generator, and having conducting surfaces, external to said casing, for making contact with said tissue; and
an impedance modulator, comprising an impedance probe, for applying electrostimulation, which is proportional to measured tissue impedance, in synchrony with cycles of said tissue impedance;
measuring tissue impedance; and
electrostimulating said tissue, wherein the electrostimulation is proportional to measured tissue impedance, in synchrony with cycles of said tissue impedance.

44. The method of claim 43, wherein said device is adapted for ingestion.

45. The method of claim 43, wherein said device is adapted for implantation.

46. The method of claim 43. wherein said device is adapted fin insertion via a catheter.

47. The method of claim 46, and further including applying medication via said catheter.

48. The method of claim 43, wherein said power source is a galvanic cell.

49. The method of claim 43, and further including converting voltage generated by said galvanic cell to an operating voltage for said pulse generator.

50. The method of claim 43, wherein said first stimulating electrode, is physically fixed to said casing, and wherein said second stimulating electrode, is operative as a mobile electrode, wherein in a first conformation, adapted for insertion into said cavity, said second stimulating electrode is in close contact with said casing, and in a second conformation, adapted for electrostirnulation, said second stimulating electrode is detached from said casing, tethered to said pulse generator, by an insulated conducting cable

51. The method of claim 50, wherein said insulated conducting cable by which said second stimulating electrode is tethered, is designed as a spring, which is in compression in said first conformation, and which maintains said first and second electrodes apart in said second conformation.

52. A method for electrostimulating tissue of internal body cavities, comprising:
employing a device for electrostimulating tissue of internal body cavities, which includes:
a pulse generator, for generating electrostimulating pulses;
first and second stimulating electrodes, in electrical communication with said pulse generator, and having conducting surfaces, adapted for making contact with said tissue;
a casing, which forms a first chamber, which encapsulates and seals said pulse generator from body fluids; and
a power source, formed as a galvanic cell, for powering said pulse generator, powering said pulse generator by said galvanic cell; and
electrostimulating said tissue.

53. The method of claim 52, wherein said galvanic cell comprises:
an inner space, fanned as a second chamber of said casing, said second chamber having portholes for admitting body fluids to said inner space; and
a third electrod; located within said inner space, having a different electrochemical potential from said first electrode, and forming an electrical circuit with said first electrode, said circuit being closed by body fluids within said inner space.

54. The method of claim 52, wherein said galvanic cell comprises:
an inner space, formed as a second chamber of said casing, said second chamber having portholes for admitting body fluids to said inner space; and
third and fourth electrodes, located within said inner space, having a different electrochemical potential between them, and Conning an electrical circuit said circuit being closed by body fluids within said inner space.

55. The method of claim 52, and further including converting voltage generated by said galvanic cell to an operating voltage for said pulse generator.

56. The method of claim 52, wherein said device is adapted far ingestion.

57. The method of claim 52, wherein said device is adapted for implantation.

58. The method of claim 52, wherein said device is adapted for insertion via a catheter.

59. The method of claim 58, and further including applying medication via said catheter.

60. The method of claim 52, wherein said first stimulating electrode, with which said galvanic cell is formed, is physically fixed to said casing, and wherein said second stimulating electrode, is operative as a mobile electrode, wherein in a first conformation, adapted for insertion into said cavity, said second stimulating electrode is in close contact with said casing, and in a second conformation, adapted for electrostimulation, said second stimulating electrode is detached from said casing, tethered to said pulse generator, by an insulated conducting cable.

61. The method of claim 60, wherein said insulated conducting cable by which said second stimulating electrode is tethered, is designed as a spring, which is in compression in said first conformation, and which maintains said first and second electrodes apart in said second conformation.

62. The method of claim 52, wherein said device further includes an impedance modulator, for applying electrostimulation, which is proportional to measured tissue impedance, in synchrony with cycles of said tissue impedance.
